(19) United States
(12) Patent Application Publication
(10) Pub. No.: US 2026/0232201 A1
(43) Pub. Date: Aug. 13, 2026

(54) SYSTEM AND METHOD FOR CONSCIOUSNESS, UNCONSCIOUSNESS, OR SENTIENCE MEASUREMENT OR ASSESSMENT

(71) Applicant: The Methodist Hospital, Houston, TX (US)

(72) Inventor: SANTOSH ANAND HELEKAR, Sugar Land, TX (US)

(21) Appl. No.: 19/154,630

(22) PCT Filed: Feb. 8, 2024
(86) PCT No.: PCT/US2024/015021

§ 371 Date: Aug. 7, 2025

Related U.S. Application Data
(60) Provisional application No. 63/483,877, filed on Feb. 8, 2023.

(51) Int. Cl. A61B 5/00; A61B 5/16

(52) U.S. Cl. CPC A61B 5/0082; A61B 5/165; A61B 5/4064; A61B 5/4821; A61B 5/6803; A61B 5/7264; A61B 5/7278; A61B 5/742; A61B 2503/40; A61B 2560/0443; A61B 2562/0238; A61B 2562/04

(57) ABSTRACT

An exemplary system and method are disclosed that can measure the level of consciousness and unconsciousness of a person or animal using an apparatus that is sensitive to light-wave interference or divergence/diffraction measurement. The exemplary system and method can be employed to decipher and map brain activity patterns that cause conscious experiences to a measured unit.

### RELATED APPLICATION

This PCT claims priority to, and the benefit of, U.S. Provisional Patent Application No. 63/483,877, filed Feb. 8, 2023, which is incorporated by reference herein in its entirety.

### BACKGROUND

There are currently no objective methods to quantify or measure human or animal consciousness, though the implication of such measure would have great utility, including in the field of medicine.

### SUMMARY

An exemplary system and method are disclosed that can measure the level of consciousness and unconsciousness of a person or animal (also termed here as Sentiometry) using an apparatus that is sensitive to light-wave interference or divergence/diffraction measurement. The exemplary system and method can be employed to decipher and map brain activity patterns that cause conscious experiences to a measured unit, referred to herein as a “Qualiagraphy.”

In some embodiments, the system includes low power laser light emitting diode, (LED), a row or array of light sensors or a light-sensitive screen, and corresponding electronics to measure the current induced by the light to be stored in a computerized device (e.g., storage device). The system and method determine deviations in the amplitudes of these currents over short intervals of time over a continuous recording. The deviations may be displayed on a display screen by a software program or subsequently employed in medical applications (e.g., control of anesthesia). The system may include a single or double slit partition to generate the light-wave interference, or it may be configured without the slit and the measurement is of the divergence or diffraction.

The second apparatus is a miniaturized Mach-Zehnder interferometer in which, instead of a slit partition, there are two beam splitters and two mirrors. Two light sensors are positioned at a fixed distance perpendicular to each other. The currents from both sensors are measured, and the ratio (second splitter divided by the first splitter) of the two amplitudes is computed instead of their deviations. Measurement of the level of consciousness involves at least two interference or divergence/diffraction apparatuses or two interferometers—one placed on a cap on the head or within 5 cm of the head (test apparatus) and the other placed at a distance of at least 90 cm (reference apparatus).

A consistently large difference in the deviation values between the interference or divergence/diffraction apparatuses or a substantially larger ratio of the test interferometer compared to the reference interferometer is a measure (Q-metric) of the level of consciousness. Mapping of brain activity patterns that code for conscious experiences involves randomly repeated presentations of sensory stimuli and averaging the current amplitudes recorded by one or more test apparatuses (on the cap) over numerous such presentations. This system of apparatuses and associated methods have applications in anesthesiology, diagnosis of altered states of consciousness, diagnosis of disorders of consciousness and brain death, sleep medicine, diagnosis of psychiatric conditions, detection of consciousness in non-human organisms, and mapping of the neural code of conscious experience. The invention of the apparatuses and methods described here resulted from the need to test the predictions of a hypothesis developed by Santosh Helekar to account for the physical basis of consciousness.

In an aspect, a method for consciousness, unconsciousness, or sentience measurement or assessment is disclosed, the method including providing a modular apparatus comprising a housing having located therein a photon source (e.g., LED or laser) and a photonic sensor or sensor assembly, wherein the photonic sensor or sensor assembly comprises at least one elongated area defining a plurality of channels or areas each configured to (i) receive interference pattern (e.g., in bands) or divergent/diffracted waves of light emitted by the photon source and (ii) measure electrical current corresponding to the received photons for each of the plurality of channels or areas; placing the modular apparatus in proximity to a person or animal (less than 3 feet from the person or animal); measuring, via electric circuitries, electrical current corresponding to the received photons for each of the plurality of channels or areas while the apparatus is in proximity to a person or animal; and outputting, via the electric circuitries or a computing device on a display, the measured electrical current or a parameter derived therefrom for each of the plurality of channels or areas, wherein the measured electrical current is employed as a measure or indicator of consciousness or sentience of the person or animal.

In another aspect, a method for consciousness, unconsciousness, or sentience measurement or assessment is disclosed, the method including providing a modular apparatus comprising a photon source (e.g., LED or laser) and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas; placing the modular apparatus in proximity to a person or animal; measuring, via electric circuitries, electrical current corresponding to the received photons for each of the plurality of channels or areas while the apparatus is in proximity to a person or animal; determining, by a processor, a consciousness or sentience measure based on the measured electrical current or a parameter derived therefrom for at least one of the plurality of channels or areas exceeds a pre-defined threshold; and outputting, via the electric circuitries or a computing device on a display, the consciousness or sentience measure, wherein the consciousness or sentience measure is employed as a measure or indicator of consciousness or sentience of the person or animal.

In another aspect, a method for consciousness, unconsciousness, or sentience assessment is disclosed, the method including providing a plurality of modular apparatuses each comprising a photon source (e.g., LED or laser) and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas, wherein the plurality of modular apparatuses are disposed at a plurality of locations on a headwear (e.g., cap); placing the plurality of modular apparatuses in proximity to a person or animal; measuring, via electric circuitries, electrical current corresponding to the received photons for each of the plurality of channels or areas while the apparatus is in proximity to a person or animal; determining, by a processor, a consciousness or sentience measure based on the measured electrical current or a parameter derived therefrom for at least one of the plurality of channels or areas exceeds a pre-defined threshold; and outputting, via the electric circuitries or a computing device on a display, the consciousness or sentience measure, wherein the consciousness or sentience measure is employed as a measure or indicator of consciousness or sentience of the person or animal.

In another aspect, a method for consciousness, unconsciousness, or sentience assessment is disclosed, the method including providing a first assembly of one or more modular apparatuses, including a first modular apparatus comprising a photon source (e.g., LED or laser) and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas, wherein first modular apparatus is disposed at a location on a wearable device (e.g., cap or other headwear) to be placed on person or animal; providing a second apparatus comprising a photon source (e.g., LED or laser) and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas; placing the first assembly comprising at least the first modular apparatus at a first position proximal to the person or animal; placing the second apparatus at a second position proximal to the person or animal, wherein the first position is closer to the person or animal than the second position; measuring, via electric circuitries, as a first measurement, electrical current corresponding to the received photons for each of the plurality of channels or areas of the first modular apparatus while the first assembly is located at the first position; measuring, via electric circuitries, as a second measurement, electrical current corresponding to the received photons for each of the plurality of channels or areas of the second apparatus while the first assembly is located at the second position; determining, by a processor, a consciousness or sentience measure based the first measurement and the second measurement; and outputting, via the electric circuitries or a computing device on a display, the consciousness or sentience measure or an indicator derived therefrom, wherein the consciousness or sentience measure or the indicator is employed as a measure or indicator of consciousness or sentience of the person or animal.

In some embodiments, the modular apparatus is disposed on a headwear.

In some embodiments, the modular apparatus includes a single or double slit located between the photon source (e.g., LED or laser) and the photonic sensor or sensor assembly to generate the interference pattern.

In some embodiments, the photonic sensor or sensor assembly of the modular apparatus is configured to measure the divergent/diffracted waves of light emitted by the photon source.

In some embodiments, the plurality of modular apparatuses includes at least one of: 2 apparatuses, 3 apparatuses, 4 apparatuses, 5 apparatuses, 6 apparatuses, 7 apparatuses, 8 apparatuses, 9 apparatuses, 10 apparatuses, between 10 and 16 apparatuses, between 16 and 32 apparatuses, and between 32 and 64 apparatuses.

In some embodiments, the method (of any of the above) further includes generating, by the processor or a different computing device, a visual output of the highest amplitude channel or a statistical parameter derived from the measurement (e.g., distribution statistics across the channels), for each of the plurality of modular apparatuses.

In some embodiments, the photonic sensor or sensor assembly comprises an array of photodiodes.

In some embodiments, the photon source comprises one or more of: an LED, a laser, or an assembly thereof.

In some embodiments, the measuring, via electric circuitries, electrical current corresponding to the received photons for each of the plurality of channels or areas comprises: determining, by the processor or hardware circuitries, band regions for the received photons for each of the plurality of channels or areas.

In some embodiments, the first modular apparatus is identical in configuration to the second apparatus.

In some embodiments, the first modular apparatus has a first configuration, wherein the second apparatus has a second configuration, and wherein the first configuration is different from the second configuration.

In some embodiments, the output is used by a clinician or a machine to adjust or administer anesthesia.

In some embodiments, the output is used by a clinician or a machine to identify or provide labels or classifications among at least brain death, comatose, vegetative, minimally conscious, and locked-in states.

In some embodiments, the output is used by a clinician or a machine to (i) quantify the perception of pain or distress (e.g., in a pain study or pain treatment), (ii) quantify the level or state of sleep (e.g., in a sleep study or sleep treatment), or (iii) quantify hallucination states, moods, beliefs, recurrent thoughts or recurrent feelings (e.g., in a psychiatric study or treatment).

In another aspect, a device or system is disclosed comprising a modular apparatus comprising: a housing having located therein a photon source (e.g., LED or laser) and a photonic sensor or sensor assembly, wherein the photonic sensor or sensor assembly comprises at least one elongated area defining a plurality of channels or areas each configured to receive interference pattern (e.g., in bands) or divergent/diffracted waves of light emitted by the photon source; and a controller configured with electrical circuitry to measure electrical current corresponding to the received photons for each of the plurality of channels or areas.

In another aspect, a device or system is disclosed comprising a modular apparatus comprising: a miniaturized Mach Zehnder interferometer; and a controller configured with electrical circuitries to measure electrical current corresponding to the received photons of the miniaturized Mach Zehnder interferometer, wherein the controller is configured to output, via the electric circuitries or a computing device on a display device, the measured electrical current or a parameter derived therefrom, wherein the measured electrical current is employed as a measure or indicator of consciousness or sentience of the person or animal.

In some embodiments, the modular apparatus includes a single or double slit located between the photon source (e.g., LED or laser) and the photonic sensor or sensor assembly to generate the interference pattern.

In some embodiments, the photonic sensor or sensor assembly of the modular apparatus is configured to measure the divergent/diffracted waves of light emitted by the photon source.

In some embodiments, the modular apparatus can be placed in proximity to a person or animal (less than 3 feet from the person or animal) to provide a measure or indicator of consciousness or sentience of the person or animal.

In some embodiments, the device or system further includes features recited in any one of the above-discussed methods.

In another aspect, a non-transitory computer-readable medium is disclosed having instructions stored thereon, wherein execution of the instructions by a processor causes the processor to (i) perform in whole or in part any one of the above-discussed methods or (ii) operate in whole or in part any one of the above-discussed device or system.

### BRIEF DESCRIPTION OF THE DRAWINGS

FIGS. 1A, 1B, 1C, 1D, 1E, 1F, and 1G each shows an example recorder and analysis system configured to measure and record consciousness, unconsciousness, or sentience of a person in accordance with an illustrative system.

FIG. 2A shows an example measured amplitude of interference bands.

FIG. 2B shows the time-series plots for the two inner bands of FIG. 2A and the two outer bands of FIG. 2A.

FIG. 2C shows examples of visualization of the recordings, e.g., that may be analyzed and presented via display.

FIGS. 3A, 3B, and 3C show a first prototype device for the double-slit interference experiment.

FIGS. 3D and 3E show a second prototype device for the double-slit interference experiment.

FIGS. 3F, 3G, and 3H show a third prototype device with differing configurations that measures diverging/diffracting waves.

FIGS. 4A-4G show experimental results acquired by a prototyped Sentiometer device. FIG. 4A shows a device placed inside an enclosure in proximity with live mice. FIG. 4B shows a time series recording of a single photodiode channel response. FIG. 4C shows normalized recorded measurements acquired from two canine subjects at 0 cm (touching subject) at two-time intervals. FIGS. 4D and 4E show raw measurements of a recorder placed next to five awake mice at two locations (next to the animals and about 11 feet away). FIGS. 4F and 4G show raw measurements of a recorder placed next to five awake mice at two locations.

FIGS. 5A-5E each shows, for 4 respective subjects, normalized recorded measurements acquired at 4 different distances (0 cm (touching subject), 30 cm away, 90 cm away, and 180 cm away, see FIG. 5E) and at four-time intervals.

FIGS. 6A-6H show measurements during sleep. FIG. 6A shows a measurement acquired in the empty room having one subject for a portion of the measurement. FIG. 6B shows a measurement acquired in the empty room with 1 subject sleeping throughout a nighttime recording (10 hours). FIG. 6C shows a measurement acquired in the empty room with another subject sleeping throughout a nighttime recording (10 hours). FIG. 6D shows a similar nighttime recording with two sleeping subjects. The second subject falls asleep and wakes up during the recording while the first subject is still asleep. FIG. 6E shows a measurement acquired in the empty room with 2 subjects sleeping throughout a nighttime recording (10 hours). FIGS. 6F, 6G, and 6H show three measurements acquired at a similar time of the day with the recorder being placed in a laboratory.

FIG. 7A shows a measurement acquired of a person with the device held at different orientations, with the device oriented perpendicular to the ground and parallel to the ground.

FIG. 7B shows a measurement acquired of a person for a period of time to determine the time to saturation of the measurement.

FIG. 7C shows measurements acquired of a person with the recorder device for a set of activities (reading and watching a video).

FIGS. 8A-8F show measurements using divergent/diffracted-wave measurement hardware to measure and record consciousness, unconsciousness, or sentience of a person in accordance with an illustrative system. FIG. 8A shows the prototype configured as a divergent/diffracted-wave measurement system. FIG. 8B shows measurements acquired of a person with the device modified (double slit included or no double slit). FIG. 8C each shows normalized recorded measurements acquired at 5 different locations of a person at the top, forehead, right, left, and back of the head. FIG. 8D shows a measurement by the recorder being placed in a generally vacated laboratory. FIG. 8E shows a measurement with the laser diode disabled to illustrate the measurements is associated with the laser source. FIG. 8F shows a measurement by two recorders, one with a double-slit configuration and one without, being placed within 10 cm of a single animal (mice).

FIGS. 9A-9B show measurements from different type of animals for a response. FIG. 9A shows measurements acquired from a person (primate) and an animal (rodent). FIG. 9B shows measurements acquired from invertebrate animals.

FIGS. 9C-9E show measurements following death. FIG. 9C shows a measured response to 20-min exposure. The response appears to be inverted 2 hours after induction of euthanasia. FIG. 9D shows the measured sentiometric response from an animal (euthanized mice) with a decapitated head and body. In FIG. 9D, the baseline measurement shows an inversion of the response being observed with respect to exposure of the head (before death). FIG. 9E shows a measured sentiometric response from an excised brain of an animal.

FIG. 10 shows a measured sentiometric response induced by a 30-min exposure of the hand to a sensor module of a sentiometer placed 15 cm from the side of the body.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED SPECIFICATION

Each and every feature described herein, and each and every combination of two or more of such features, is included within the scope of the present invention provided that the features included in such a combination are not mutually inconsistent.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entirety and to the same extent as if each reference was individually incorporated by reference.

### Example System #1

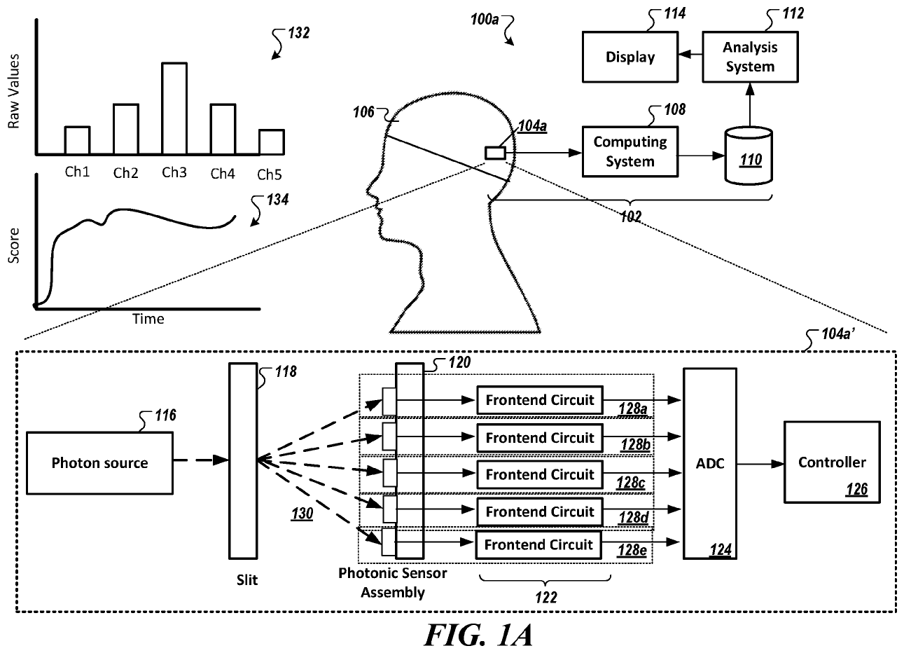

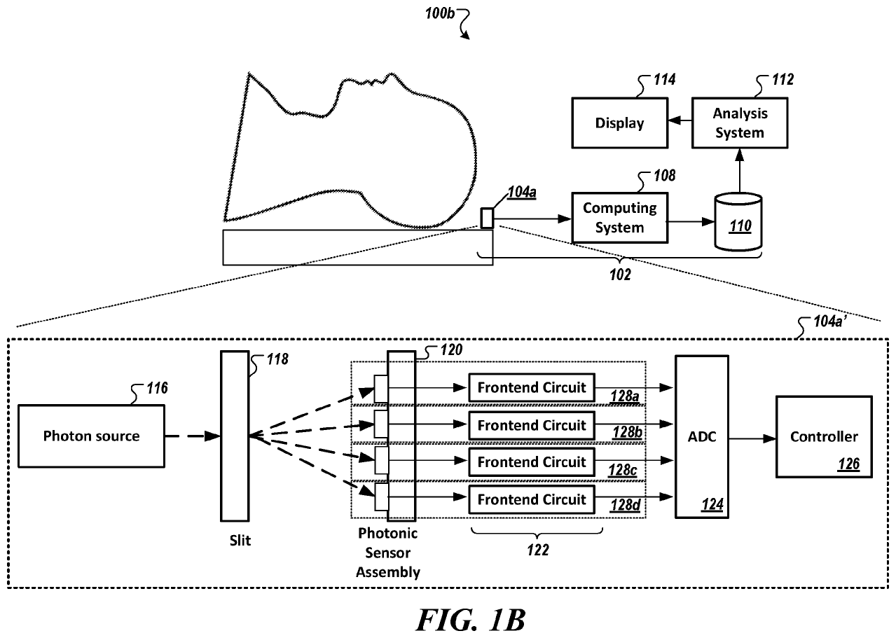

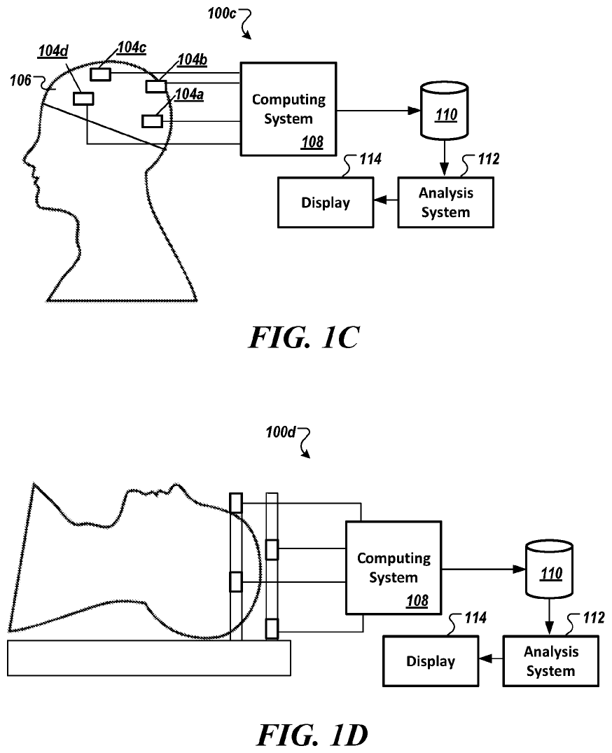

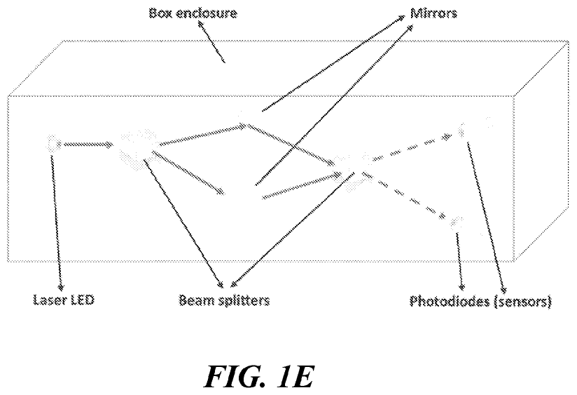

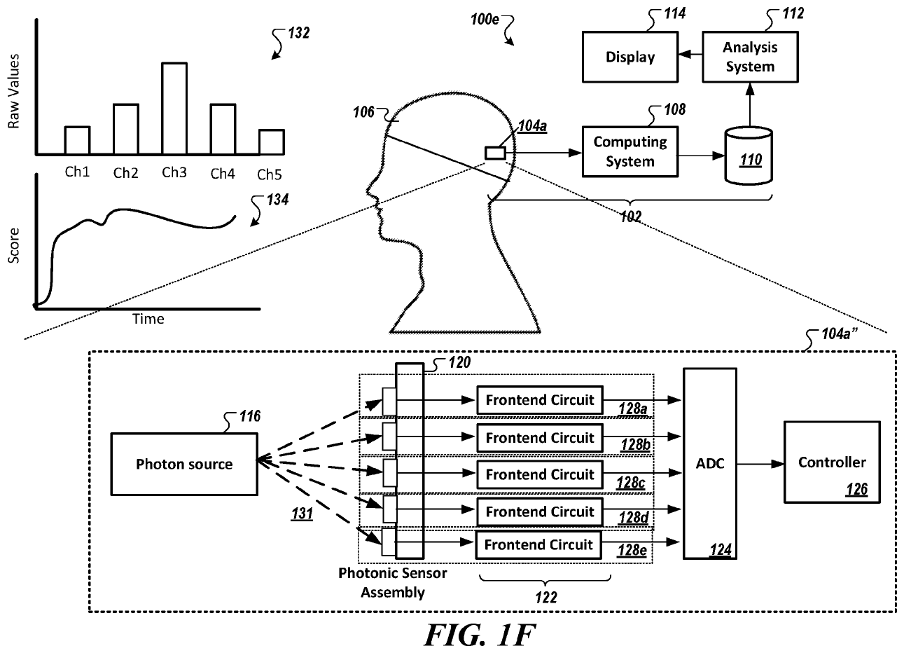

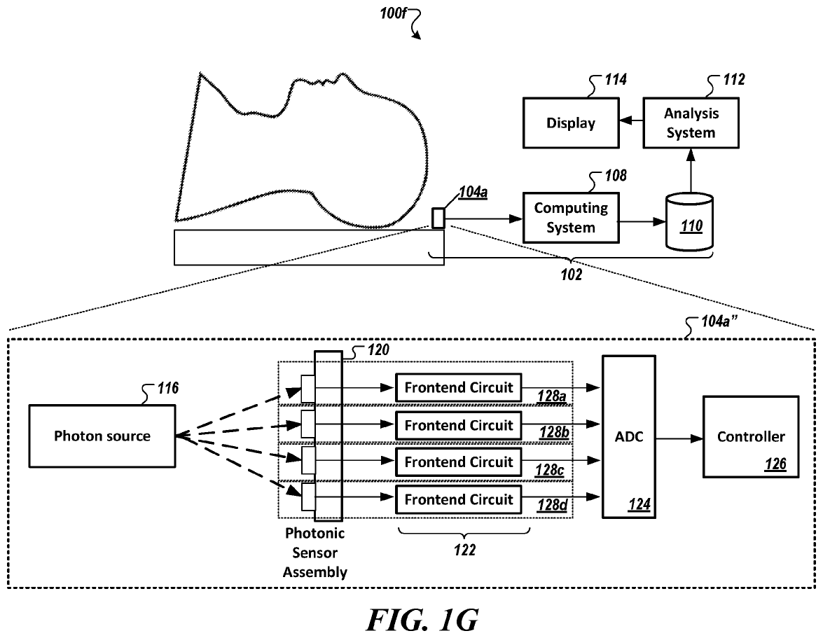

FIGS. 1A, 1B, 1C, 1D, 1E, and 1F each shows an example recorder and analysis system 100 (shown as 100a, 100b, 100c, 100d, 100e, and 100f respectively) configured to measure and record consciousness, unconsciousness, or sentience of a person in accordance with an illustrative system.

In the example shown in FIGS. 1A, 1B, 1C, 1D, 1E, and 1F, the recorder and analysis system (e.g., 100a, 100b, 100e, 100f) includes a measurement system 102 comprising a recorder device 104 (shown as 104a), e.g., disposed on a wearable cap 106 to be worn by a person or placed in proximity to a person's head. The recorder device 104 is connected to a computing device 108 having a data store 110. The recorder and analysis system 100a includes an analysis system 112 configured to retrieve a recording from the data store 114 to generate a report, e.g., on a display device 116, that shows a measure or indicator of consciousness or sentience of the person or animal.

The recorder device 104 is a quantum-effect sensitive device or quantum-effect observer that can observe, over a period of time, e.g., the effects of quantum wave function collapse or decoherence, to provide a measure of consciousness, unconsciousness, or sentience of a person. The recording and/or measurement can be used to assess the state of being awake/sedated, functional cognitive, or noncognitive assessment for a person, e.g., a coma patient, a person sleeping, and the like. Indeed, the recorder device 104 has utility in various medical and clinical applications as well as in neuroscience research, among others. The recorder device 104 may have utility for trauma and mental health treatments, law enforcement, and security intelligence applications.

The exemplary system and method can be used (i) to measure the depth of consciousness under general anesthesia in the operating room, (ii) to discern the extent to which an unresponsive subject in the intensive care unit is conscious, e.g., allowing differentiation between brain death, comatose, vegetative, minimally conscious and locked in states, (iii) to determine the frequency and nature of dreams in sleep studies, potentially useful in the diagnosis of psychoses, (iv) to quantify the intensity of pain and distress, (v) to determine the frequency and nature of disordered thoughts, moods, feelings, beliefs and hallucinations in psychiatric conditions, (vi) to detect whether a prematurely born baby or a fetus in utero is conscious or not, (vii) to discover whether a living organism on the evolutionary ladder is conscious or not; and (viii) to decipher the entire neural-quantum code of sensory, affective and cognitive qualia, akin to the mapping of the genomes of humans and other organisms.

As noted above, in the example of FIG. 1B, the recorder device 104a is fixably coupled to a structure 107 (e.g., bed, chair) and is placed in proximity to the person. The structure maintains constant proximity/distance between the recorder device 104a and the person (e.g., head), e.g., less than 5 cm.

Referring to FIG. 1A, the recorder device 104a′ is configured as a slit diffraction device that implements a single or double slit quantum-physics experiment. The recorder device 104a′ includes a photon source (e.g., LED or laser) 116, a slit partition (e.g., single-slit or double-slit) 118, and a photonic sensor or sensor assembly 120 that couples to electronics 122 (shown as “Frontend Circuit” 122) that amplifiers and conditions the measured photon that is received at the photonic sensor or sensor assembly 120. The measured signal is converted to a digital value via an analog-to-digital converter 124 that couples to a controller 126. The photonic sensor or sensor assembly 120 includes an elongated area defining a plurality of channels (shown as 128a, 128b, 128c, 128d, 128e), each configured to receive interference pattern 130 (e.g., in bands) of light emitted by the photon source 116 and traveled through the slit partition 118. The front-end circuitries 122 and ADC(s) 124 are configured with electrical circuitry to measure electrical current corresponding to the received photons for each of the plurality of channels 128.

Diagram 132 shows a plot of the amplitude of the measured current for each channel 128, at one instance in time, of the photonic sensor or sensor assembly 120 corresponding to the measured photons. Diagram 134 shows a time-series plot of the deviation of the measurement normalized. To generate the time-series plot, the baseline value (i.e., an initial point where there is no consciousness) can be determined and subtracted from the raw signal amplitude. The result is an inverted plot, e.g., as shown in diagram 134.

### Example System #2

FIG. 1C shows the example recorder and analysis system 100c configured with an array of recorder devices 104 (shown as 104a, 104b, 104c, 104d) in accordance with an illustrative system. The number of recorder devices 104 in the array may include 2, 3, 4, 5, 6, 7, 8, 9, 10, 11, 12, 13, 14, 15, 16, 17, 18, 19, 20. In some embodiments, the number of recorder devices 104 is greater than 20.

FIG. 1D shows another example recorder and analysis system 100d configured with an array of recorder devices 104 (shown as 104a, 104b, 104c, 104d) in accordance with an illustrative system. In FIG. 1D, the array of recorder devices 104 is mounted onto a structure that surrounds the person's head with the recorder devices while maintaining the recorder devices 104 within a pre-defined distance from the person's head.

### Example System #3

FIG. 1E shows another example recorder and analysis system 100e configured with a recorder device 104 (104e) configured as a Mach-Zehnder-type Interferometer in accordance with an illustrative system. A miniaturized Mach-Zehnder-type Interferometer may be implemented in the configurations shown in FIGS. 1A-1D.

### Example System #4

FIG. 1F shows the example recorder and analysis system 100e configured with an array of recorder devices 104 in accordance with an illustrative system.

Referring to FIG. 1F, the recorder device 104a″ is configured as a diffraction device that is configured to measure divergent/diffracted waves of light emitted by a photon source. The recorder device 104a″ includes a photon source (e.g., LED or laser) 116 and a photonic sensor or sensor assembly 120 that couples to electronics 122 (shown as “Frontend Circuit” 122) that amplifiers and conditions the measured photon that is received at the photonic sensor or sensor assembly 120. The measured signal is converted to a digital value via an analog-to-digital converter 124 that couples to a controller 126. The photonic sensor or sensor assembly 120 includes an elongated area defining a plurality of channels (shown as 128a, 128b, 128c, 128d, 128e), each configured to receive divergent/diffracted pattern 131 (e.g., in bands) or divergent/diffracted waves of light emitted by the photon source 116. The front-end circuitries 122 and ADC(s) 124 are configured with electrical circuitry to measure electrical current corresponding to the received photons for each of the plurality of channels 128.

Diagram 132 shows a plot of the amplitude of the measured current for each channel 128, at one instance in time, of the photonic sensor or sensor assembly 120 corresponding to the measured photons. Diagram 134 shows a time-series plot of the deviation of the measurement normalized. To generate the time-series plot, the baseline value (i.e., an initial point where there is no consciousness) can be determined and subtracted from the raw signal amplitude. The result is an inverted plot, e.g., as shown in diagram 134.

FIG. 1G shows the example recorder and analysis system 100f configured with an array of recorder devices 104 in accordance with an illustrative system.

As noted above, in the example of FIG. 1G, the recorder device 104a″ is fixably coupled to a structure 107 (e.g., bed, chair) and is placed in proximity to the person. The structure maintains constant proximity/distance between the recorder device 104a and the person (e.g., head), e.g., less than 5 cm.

### Example Measurement

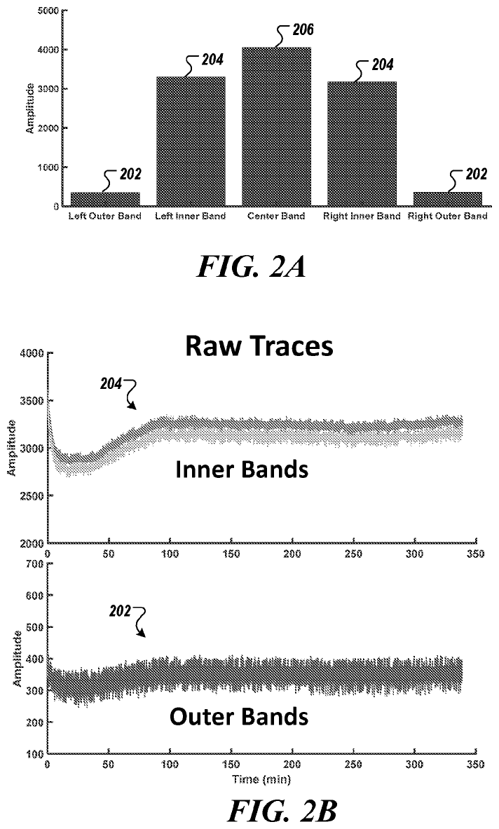

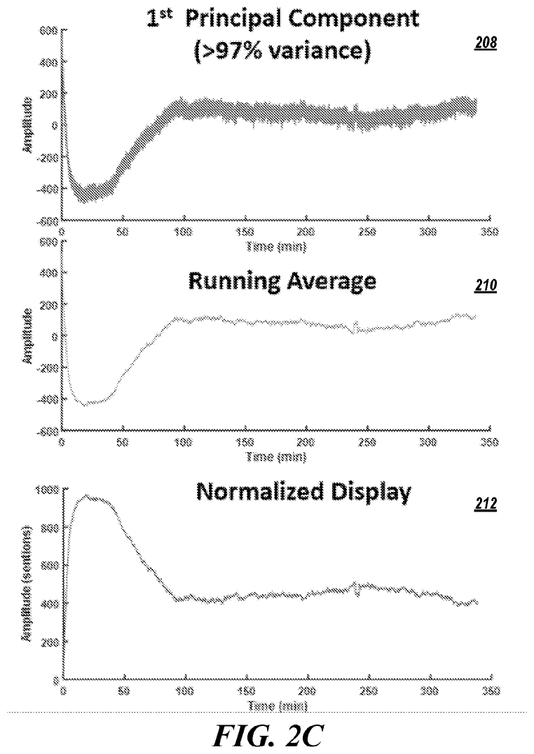

FIG. 2A shows an example measured amplitude of interference bands. In FIG. 2, the measured amplitude is for five channels: left and right outer bands 202, left and right inner bands 204, and center band 206. The y-axis shows the raw measured values (bits) (12 bits).

FIG. 2B shows the time-series plots for the two inner bands of FIG. 2A and the two outer bands of FIG. 2A.

FIG. 2C shows examples of visualization of the recordings (in diagrams 208, 210, and 212), e.g., that may be analyzed and presented via display 114.

Table 1 shows the operations to generate the visualizations of FIG. 2C.

TABLE 1

| Visualization Operation | Description |
|---|---|
| 1^{st} Principal Component | ${w}_{( 1 )} = arg max \{ \frac{{w}^{⊤} {X}^{⊤} Xw}{{w}^{⊤} w} \}$ |
| Running Average | $\begin{matrix}{SMA}_{k} = \frac{{p}_{n - k + 1} + {p}_{n - k + 2} + … + {p}_{n}}{k} \\ = \frac{1}{k} {\sum }_{i = n - k + 1}^{n} {p}_{i}\end{matrix}$ |
| Normalized | Determine baseline (i.e., an initial point |
| | where there is no consciousness) |
| | Output = raw signal amplitude - baseline |

Examples of usage of the exemplary system and method are provided in Table 2.

TABLE 2

| Example Utility | Description |
|---|---|
| Hospital Emergency Room | Emergency room decision on patients with no or limited state of |
| | consciousness: To discern the extent to which an unresponsive |
| | subject in the intensive care unit is conscious, e.g. allowing |
| | differentiation between brain death, comatose, vegetative, |
| | minimally conscious, and locked-in states. |
| Sleep Studies | Determine the frequency and nature of dreams in sleep studies, |
| | potentially useful in the diagnosis of psychoses. |
| Pain studies and treatment | Quantify the intensity of pain and distress. |
| Psychotic Disorders | Determine the frequency and nature of disordered thoughts, |
| | moods, feelings, beliefs and hallucinations in psychiatric |
| | conditions. |
| Fetal Consciousness | Detect whether a prematurely born baby or a fetus in utero is |
| | conscious or not. |
| Law Enforcement | Potential use in lie detection tests and deciphering thoughts, |
| | feelings, beliefs, plans and intents. |
| Research | Decipher the entire neural-quantum code of sensory, affective, |
| | and cognitive qualia, akin to the mapping of the genomes of |
| | humans and other organisms. |

### Experimental Results and Examples

A study was conducted that built the hardware and software for a prototyped slit box apparatus. Three different variations of the working prototypes have been developed.

A pilot study in normal adults is planned, as well as trial studies on anesthetized subjects in the operating room and unresponsive subjects in the intensive care unit.

The exemplary system and method resulted from the need to test two predictions of a hypothesis propounded by Santosh Helekar to account for the physical basis of consciousness or subjective experience and the unique implementation of this physics in the biology of the brain. This hypothesis is a further elaboration of a theoretical framework published by Helekar in 1999 (Helekar SA. On the possibility of universal neural coding of subjective experience. Conscious Cogn. 1999 December; 8(4}:423-46; Helekar SA. In defense of experience-coding nonarbitrary temporal neural activity patterns. Conscious Cogn. 1999 December; 8(4):455-61}.

The latest insight is derived from the long-held hypothesis that consciousness has something to do with the collapse of the wave function in quantum mechanics. In other words, it is somehow related to the materialization of particles such as photons from waves.

Roger Penrose, Stuart Hameroff, and others have hypothesized that the brain mechanism of consciousness may involve spontaneous collapse of the quantum wave function.

The final form of the hypothesis developed by Helekar is a generalization and extension of this theoretical notion to relate it to the current understanding of neural correlates and the phenomenology of consciousness, consequently generating experimentally testable predictions. It states that the underlying mechanisms producing neural activity patterns that are associated with conscious experiences or the patterns themselves generate these experiences by inducing specific patterns of collapse/decoherence of any quantum wave function in its vicinity. The temporal shapes of these collapse/decoherence patterns recapitulate the shapes of the neural activity patterns and uniquely and universally code for elements of conscious experiences long recognized as qualia. Thus, the hypothesis predicts that an apparatus that can detect the conversion of light waves into photons should be able to confirm its two main predictions, namely: 1) that a conscious experience-producing neural activity focus located within a short distance of this apparatus should convert more light waves into photons; and 2) the temporal pattern of this conversion when detected as a signal above the baseline noise should represent the code for the corresponding experience.

The study implemented the exemplary system and method using apparatuses based on the well-known double-slit experiment of quantum physics to test key predictions of a hypothesis accounting for the physical nature of consciousness, a long-standing unsolved problem. The study also considered the Mach-Zehnder interferometer.

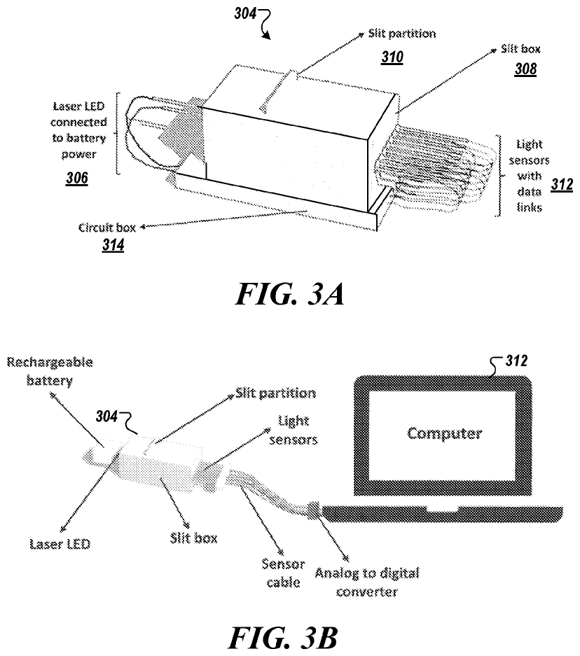

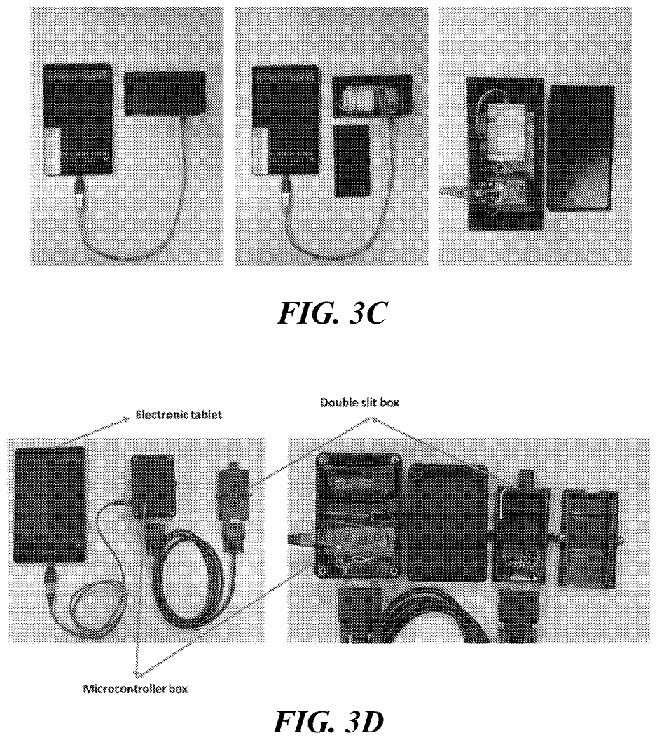

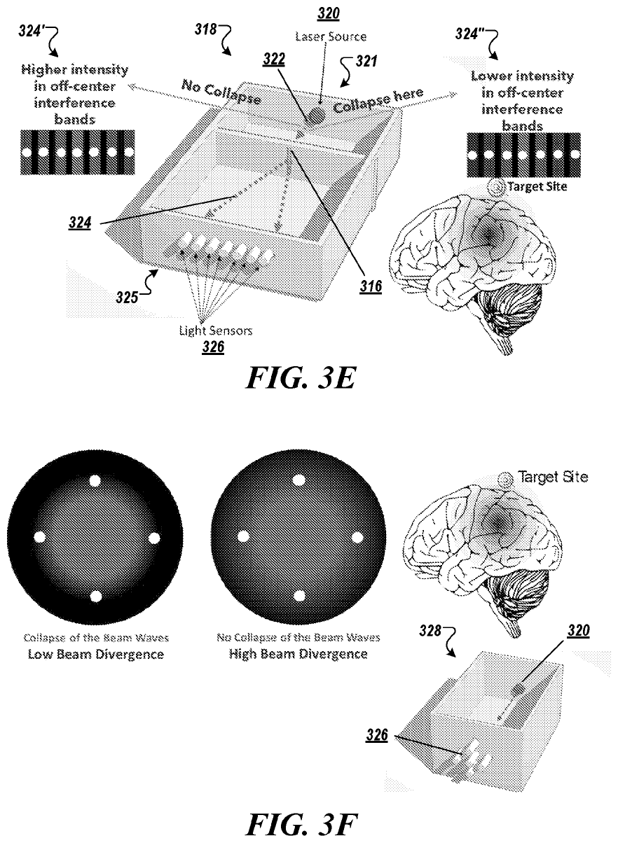

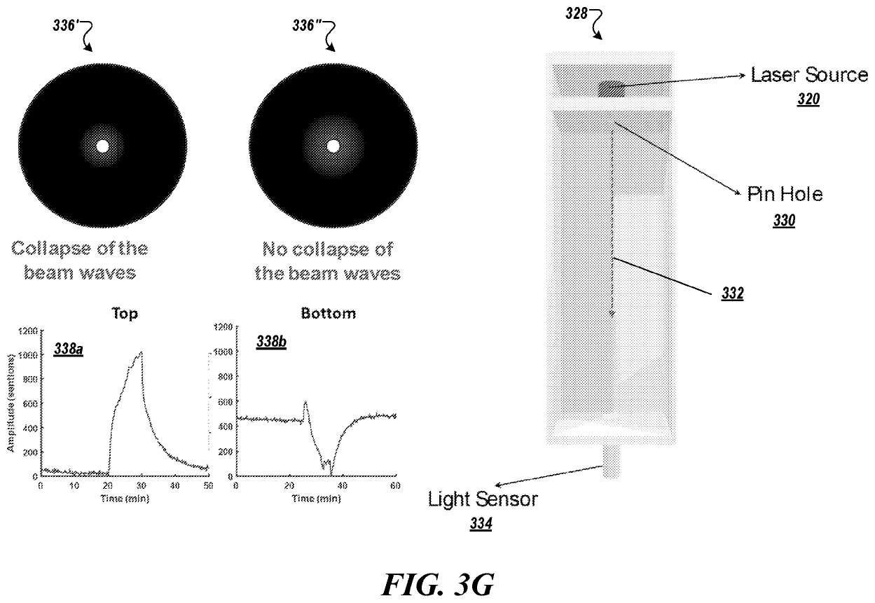

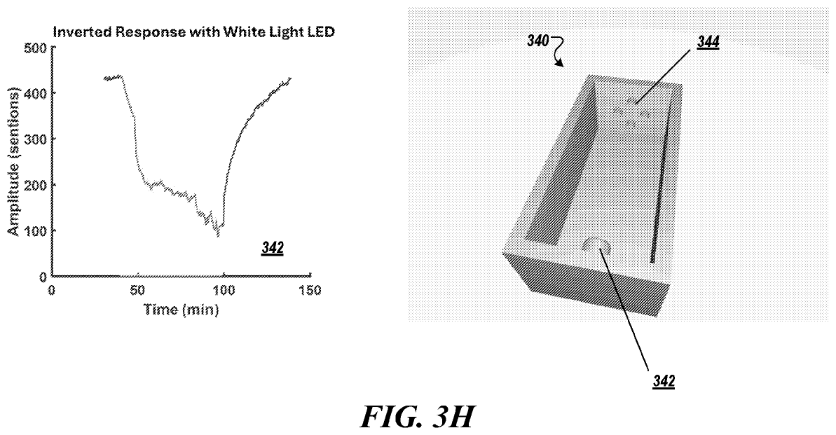

Prototype #1. FIG. 3C shows the first prototype device for the double-slit interference experiment. The device consists of a sensor unit (shown as “Slit Box” 308) containing a red dot low power laser diode, a linear array of 7 photodiodes, and a double slit partition. The sensor unit is connected to a controller box containing a microcontroller board with an onboard 10-bit analog-to-digital converter and a microprocessor uploaded with firmware that can sample photodiode currents at rates ranging from 10-500 Hz. The controller box may be connected to an electronic tablet or a computer through a USB cable. The data may be acquired and stored by a serial monitoring program at a baud rate of 9600 or stored on removable media (e.g., an SD card) installed on the microcontroller board. The first apparatus 304 (FIGS. 3A and 3B) includes a box 308 (shown as “Slit Box” 308) in which a low power laser light emitting diode (LED) 306 emits light that is projected through a single or double slit partition 310 on to a row of light sensors 312 (i.e., photodiodes) to produce a fringe pattern consisting of bright and dark bands. The study considered a light-sensitive screen as an alternative to the light sensor. An electronic circuit 314 (shown as “Circuit Box” 314) measures the currents induced by the light in each bright or dark band on the left and the right side of a central bright band. The amplitudes of these currents were converted into digital form and were either stored on a computer connected to the apparatus through an analog-to-digital converter or on removable digital media such as a micro-secure digital card when the apparatus was employed in a standalone configuration. Deviations of the amplitudes of these currents over short intervals of time during continuous recording were used to compute a single statistical value, such as the mean or the sum of the maximum range of deviations, and displayed on a display screen by a software program.

Prototype #2. FIG. 3D shows a second prototype device for the double-slit interference experiment. The second prototype device is configured to acquire measurements at 12 bits at 100 Hz. FIG. 3E shows a diagram for the prototype device of FIG. 3D for the double-slit interference experiment.

In FIG. 3E, the device includes a double slit partition 316 in the sensor module 318. A laser source 320, located at the first end 321 of the sensor module, provides a laser beam 322 through the double slit partition 316 that creates an interference pattern 324 (see also 324′ and 324″) at the second end 325, configured with a set of photodiodes 326 (shown as “Light Sensor” 326). The interference pattern 324′ shows a higher intensity measurement in the off-center interference band when no collapse/decoherence is present, e.g., due to the hypothetical mechanism related to conscious experience. The interference pattern 324″ shows a lower intensity measurement in the off-center interference band, indicating a collapse/decoherence is present, e.g., due to the hypothetical mechanism related to conscious experience.

The device can be placed in proximity to the peri-cranial or peri-somatic as a measurement site for predicted effects on off-center interference bands.

Prototype #3. FIG. 3F shows a third prototype device that measures diverging/diffracting waves. This configuration has no double slit partition in the sensor module 328 and simply measures the sampling of the diverging/diffracting waves. The sensor module 328 includes a laser source 320 and a set of photodiodes 326 to detect the degree of divergence/diffraction of the beam. A measurement of a low beam divergence/diffraction indicates a collapse/decoherence of the beam wave, e.g., due to the peri-somatic effect. A measurement of a high beam divergence/diffraction indicates no collapse/decoherence of the beam wave, e.g., due to the peri-somatic effect.

Prototype #4. FIG. 3G shows another configuration of the sensor module 328 of FIG. 3F that can measure diverging/diffracting waves. The module 328 includes a pinhole aperture 330 to which the laser source 320 is positioned. The laser light 332 from the laser source 320 passes through the pinhole aperture 330. The sensor module 328 includes a single central photodiode 334 to sample the light 332 emerging from the pinhole 330. The response 336 (shown as 336′ and 336″) can change shape in the presence of a collapse/decoherence of the beam (336′) or no collapse/decoherence of the beam (336″), e.g., due to the peri-somatic effect. Plots 338 (shown as 338a, 338b) show the measurement at two orientations of the sensor module 328.

Prototype #4. FIG. 3H shows another configuration of a sensor module 340 configured to measure diverging/diffracting waves. The sensor module 340 employs a white light LED 342, instead of a laser LED (e.g., 320), that generates a light that is measured by photodiodes 344 to measure the peri-somatic effect. Because it can detect a response, this suggests the detection of the peri-somatic effect does not appear to depend on a specific wavelength. Plot 342 shows the response appears to be inverted with the highlighted region depicting the duration of exposure.

Mach Zehnder interferometer. The study also considered a second apparatus, as another embodiment, as a miniaturized Mach Zehnder interferometer in which, instead of a slit partition, two beam splitters and two mirrors may be used (FIG. 2). The light from the laser diode may pass through the first beam splitter and split into two perpendicular beams. Each beam may then be reflected by a mirror, and the two reflected beams are passed through the second beam splitter. The arrangement of the splitters and mirrors could be such that light waves combine to reform the original beam in the second beam splitter. The combined beam is then detected by one of two light sensors (e.g., photodiodes) that are positioned at a fixed distance perpendicular to each other if the waves do not collapse/decoherence into photons en route to the splitters. If this collapse/decoherence occurs due to the hypothetical mechanism related to conscious experience as predicted, then the second sensor could also detect a light beam. Therefore, currents from both sensors are measured, and the ratio of the two amplitudes may be computed to detect the experience-related collapse/decoherence.

Methodology. The study measured the level of consciousness (termed here as “Sentiometry”) using the two slit apparatuses. In the study, the apparatus was placed near a subject's head within 5 cm of the head (test apparatus). Experiments were conducted at various distances and in proximity up to a distance of at least 90 cm (reference apparatus).

The study compared the computed value of deviation in the amplitudes of currents generated by the test slit apparatus close to the head to the corresponding value of the distant reference apparatus at the same time points indicates that the subject is conscious and the magnitude of the mean difference between the two values is proportional to her/his level of consciousness. For interferometers, a substantially larger ratio of the test interferometer could be compared to the reference interferometer corresponds to the equivalent measure (Q-metric) of consciousness.

The study can determine the brain activity code of a conscious experience (termed as “Qualiagraphy”) at a different time and as evoked by a stimulus. The study can record measurements at different positions on a cap of the test apparatus proximal to the head variations. The recording can collect values over the duration of the stimulus. Each time it can be randomly repeated to record time-locked averaged over a large number of such presentations. The averaged waveform so obtained can represent the unique pattern of brain activity that gives rise to the conscious experience associated with that stimulus.

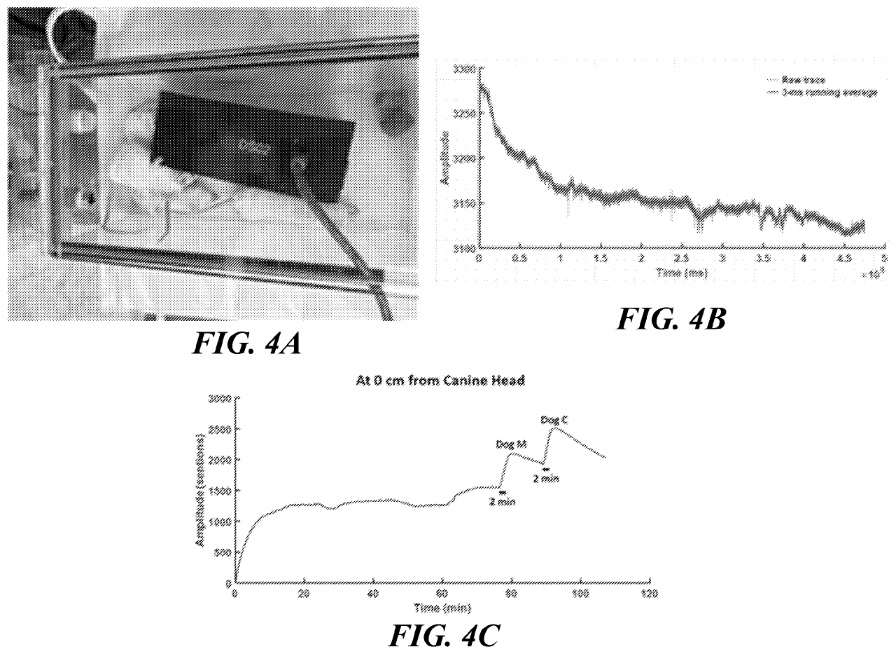

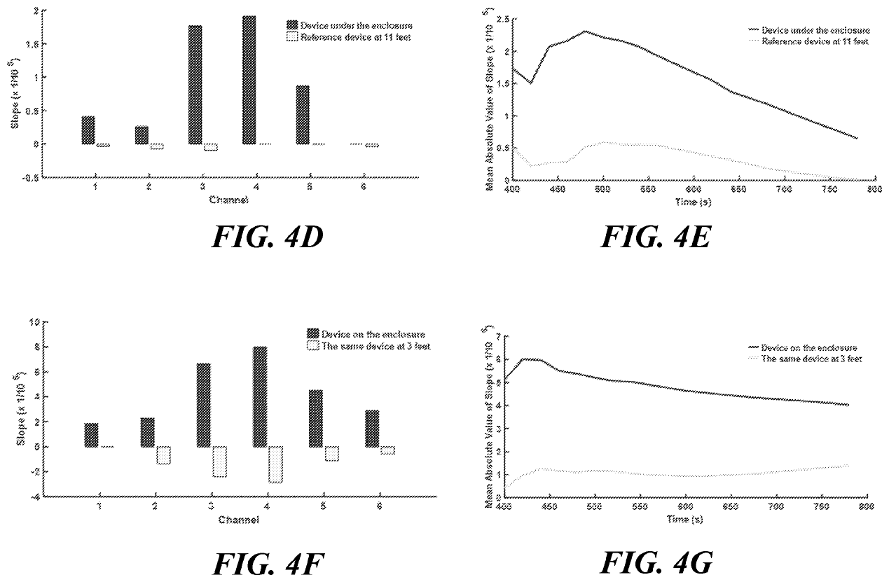

Animal Observations. FIG. 4A shows the prototyped Sentiometer device #1 placed inside an enclosure with live mice. FIG. 4B shows a time series recording of a single photodiode channel response to the light intensity at its position in the interference pattern.

FIG. 4C shows normalized recorded measurements acquired from two canine subjects at 0 cm (touching subject) at two-time intervals.

FIGS. 4D and 4E show raw measurements of a recorder placed next to five awake mice at two locations (next to the animals and about 11 feet away). In FIG. 4D, the raw measurements for the 6 channels are shown for a given time. FIG. 4E shows a time series plot for the raw measurement for one of the channels.

FIGS. 4F and 4G show raw measurements of a recorder placed next to five awake mice at two locations (next to the animals and about 3 feet away).

Human Observations. The study collected recordings of people in different experimental contexts.

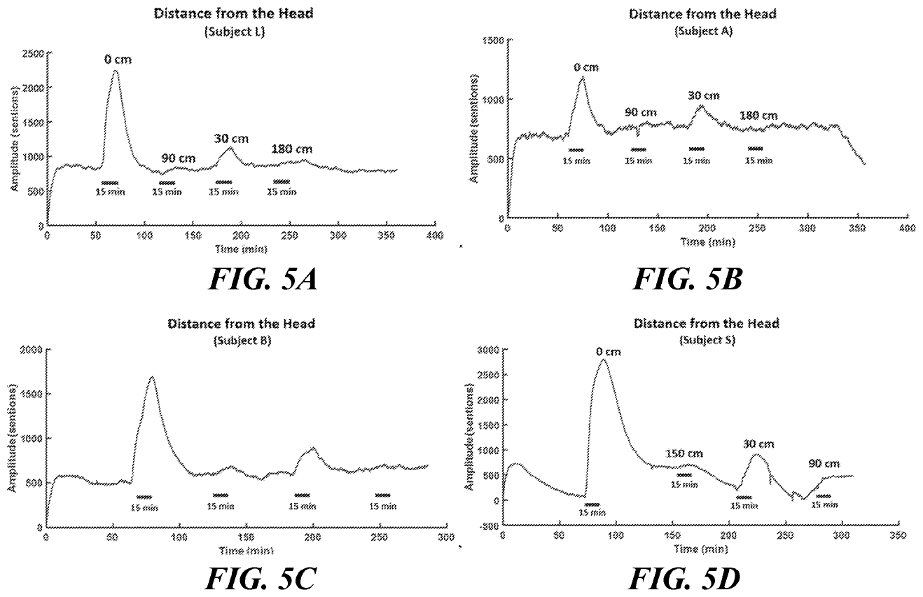

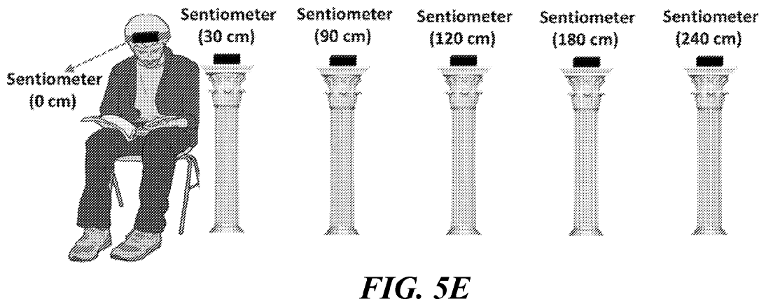

FIGS. 5A-5D each shows, for 4 respective subjects (L, A, B, S), normalized recorded measurements acquired at 4 different distances (0 cm (touching subject), 30 cm away, 90 cm away, and 180 cm away) and at four-time intervals (see FIG. 5E). It can be observed that the normalized recorded measurement maintain persistence in the data. The measurements were taken in an empty room during the daytime, with the recorder being placed at the respective locations for each of the denoted measurements. The time series plotted is the normalized trace derived, as discussed above, from the first principal component obtained by principal component analysis of data from 4 off-center channels of the device.

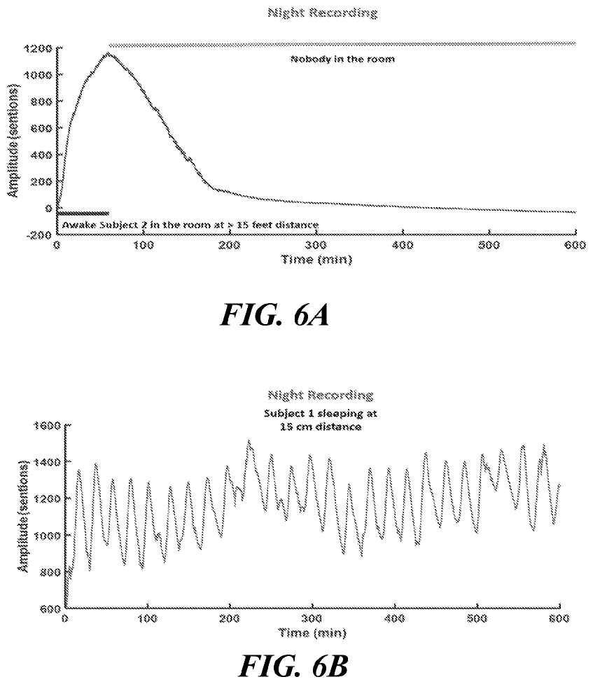

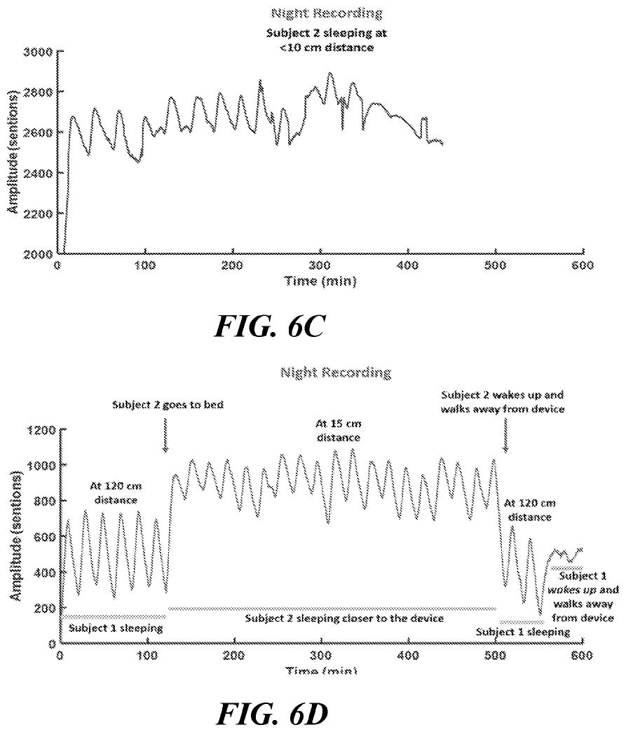

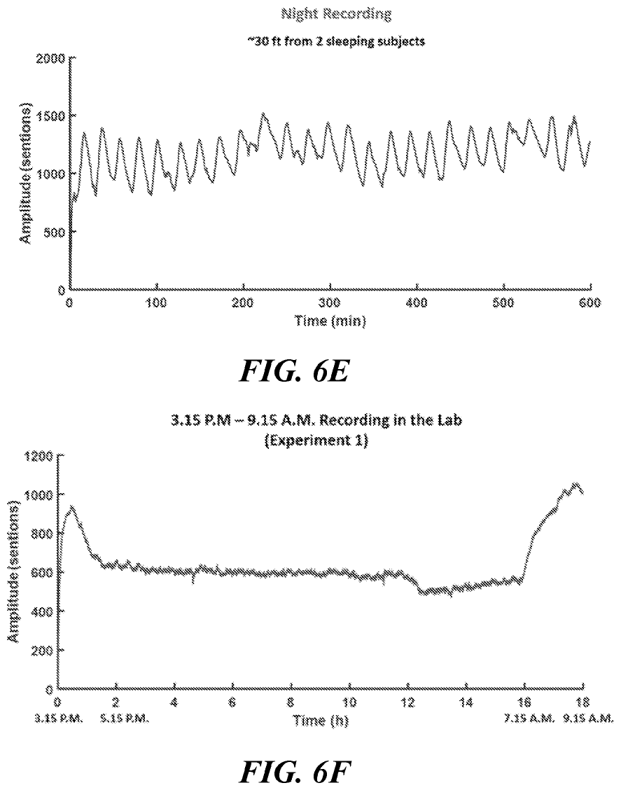

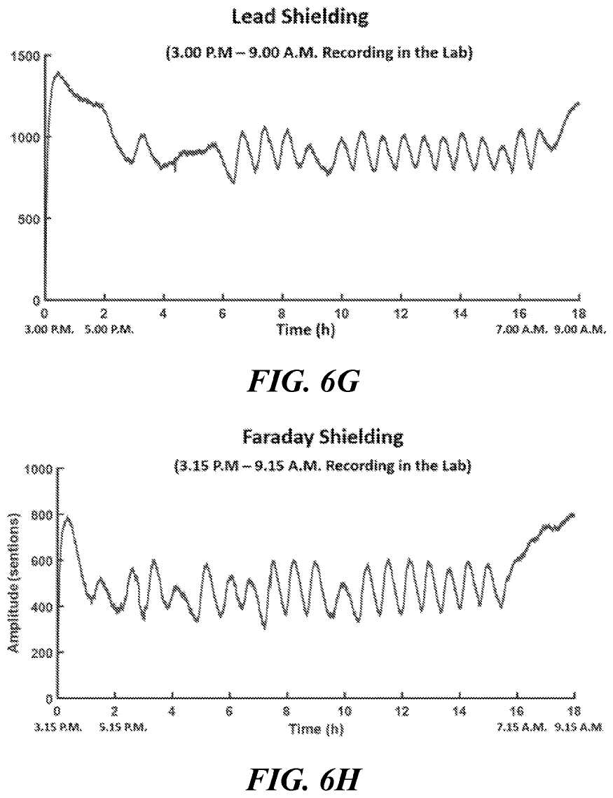

FIG. 6A shows a measurement acquired in the empty room having one subject for a portion of the measurement. FIG. 6B shows a measurement acquired in the empty room with 1 subject sleeping throughout a nighttime recording (10 hours).

FIG. 6C shows a measurement acquired in the empty room with another subject sleeping throughout a nighttime recording (10 hours). The recorder was placed less than 10 cm apart from the subject. FIG. 6D shows a similar nighttime recording with two sleeping subjects. The second subject falls asleep and wakes up during the recording while the first subject is still asleep.

FIG. 6E shows a measurement acquired in the empty room with 2 subjects sleeping throughout a nighttime recording (10 hours). The recorder was placed at about 30 feet away from each of the two subjects.

FIGS. 6F, 6G, and 6H show three measurements acquired at a similar time of the day with the recorder being placed in a laboratory. People are variously moving in and out of the space throughout the day (FIG. 6F). The second measurement (FIG. 6G) was performed with the recorder placed in a lead-shielded box to isolate the measurement of high-energy radiation effects in a hospital setting. The third measurement (FIG. 6H) was performed with the recorder placed in a faraday-shielded enclosure to isolate the measurement of radiofrequency electromagnetic field effects.

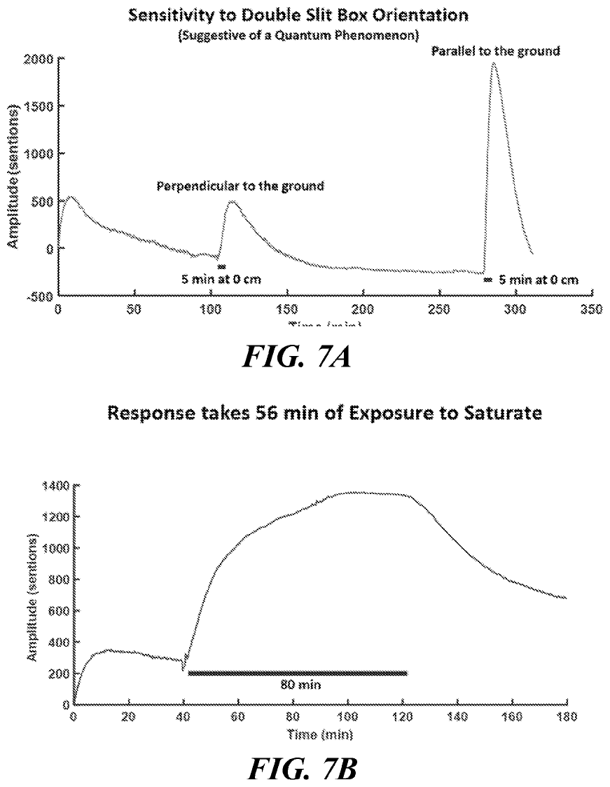

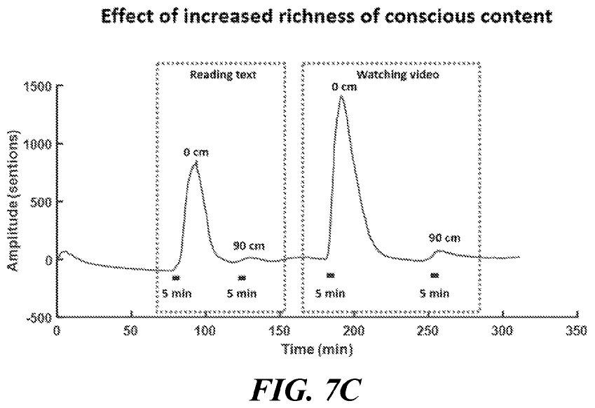

FIG. 7A shows a measurement acquired of a person with the device held at different orientations (with the device oriented perpendicular to the ground and parallel to the ground).

FIG. 7B shows a measurement acquired of a person for a period of time to determine the time to saturation of the measurement.

FIG. 7C shows measurements acquired of a person with the recorder device for a set of activities (reading and watching a video).

### Divergent/Diffracted Light Measurement Device

The study also considered a third apparatus, as another embodiment, based on a slit partition experiment but without the slit to measure divergent/diffracted light (e.g., FIGS. 3F, 3G, 3H). It was observed that this class of system was able to record similar peri-somatic measurements to those of the slit-system configuration.

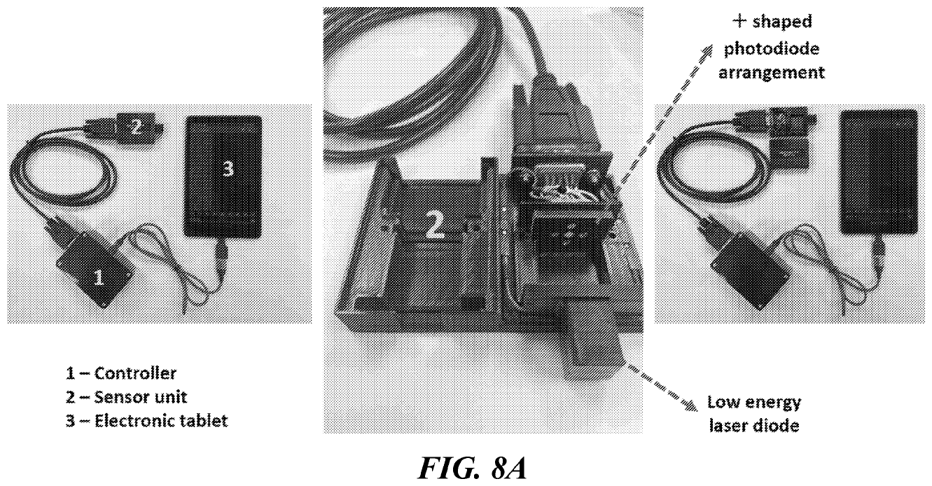

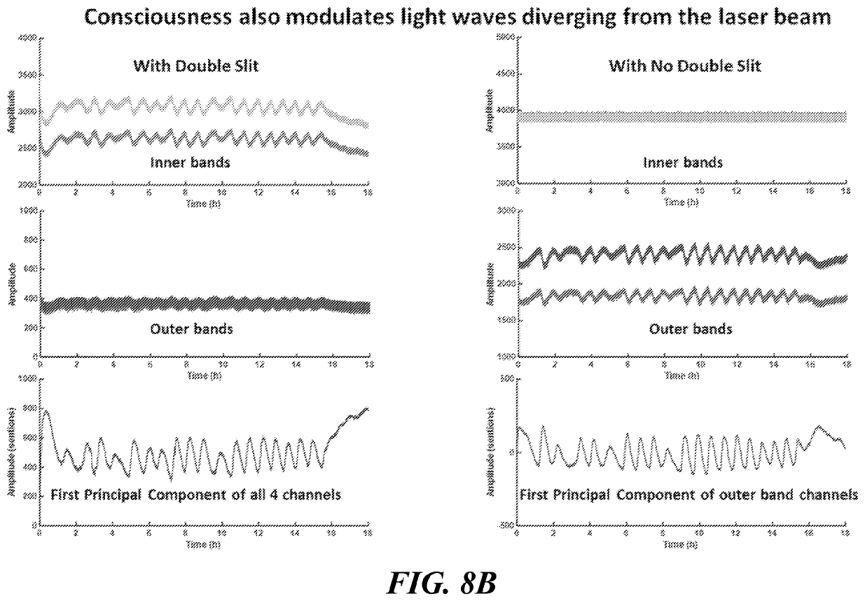

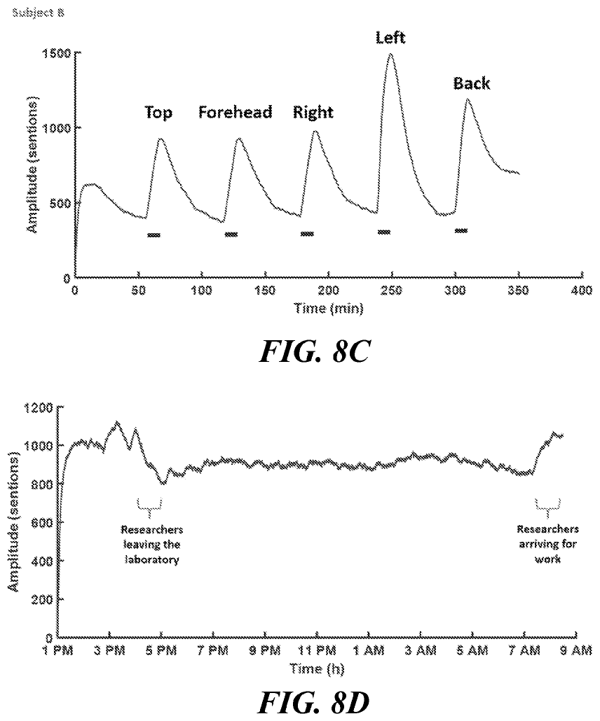

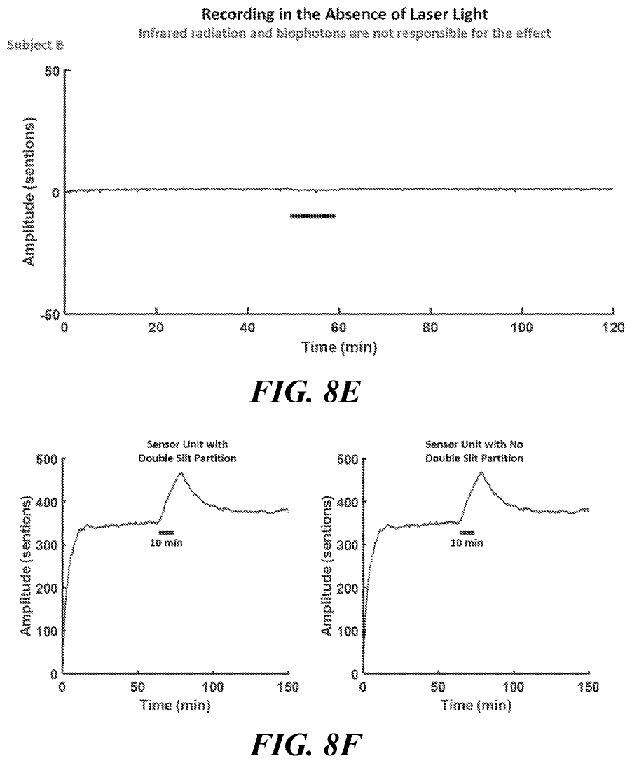

FIG. 8A shows the prototype configured as a divergent/diffracted-wave measurement system.

FIG. 8B shows measurements acquired of a person with the device modified (double slit included or no double slit).

FIG. 8C each shows normalized recorded measurements acquired at 5 different locations of a person at the top, forehead, right, left, and back of the head.

FIG. 8D shows a measurement by the recorder being placed in a generally vacated laboratory.

FIG. 8E shows a measurement with the laser diode disabled to illustrate the measurements is associated with the laser source.

FIG. 8F shows a measurement by two recorders, one with a double-slit configuration and one without, being placed within 10 cm of a single animal (mice).

### Somatic Response in Animals

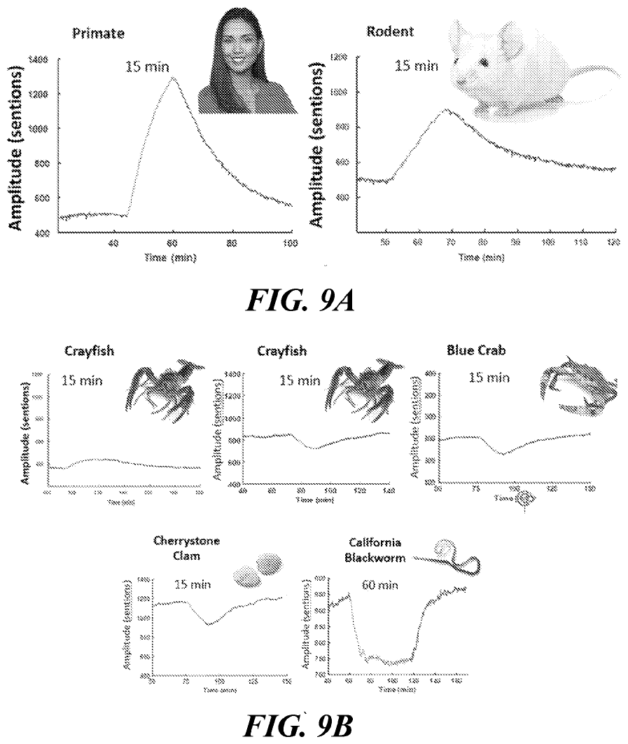

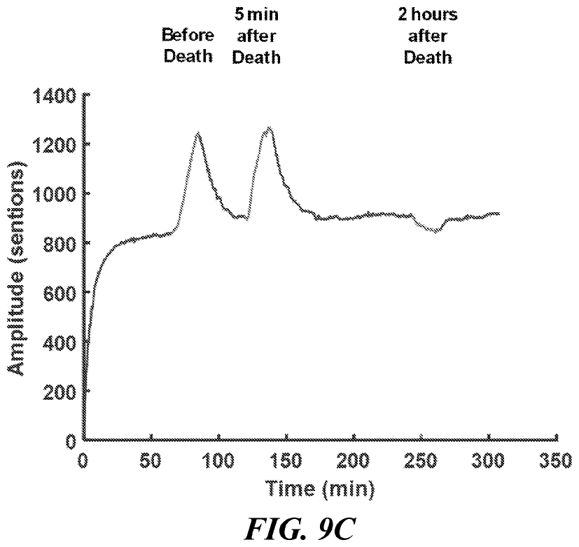

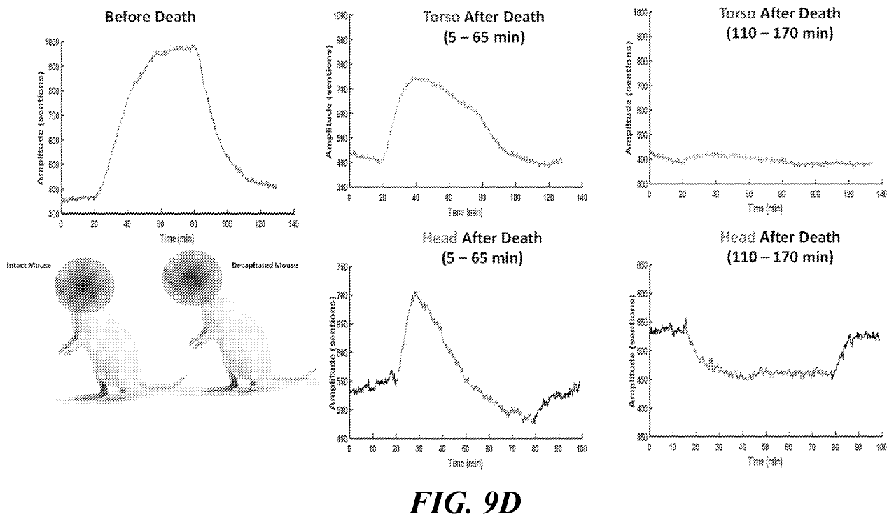

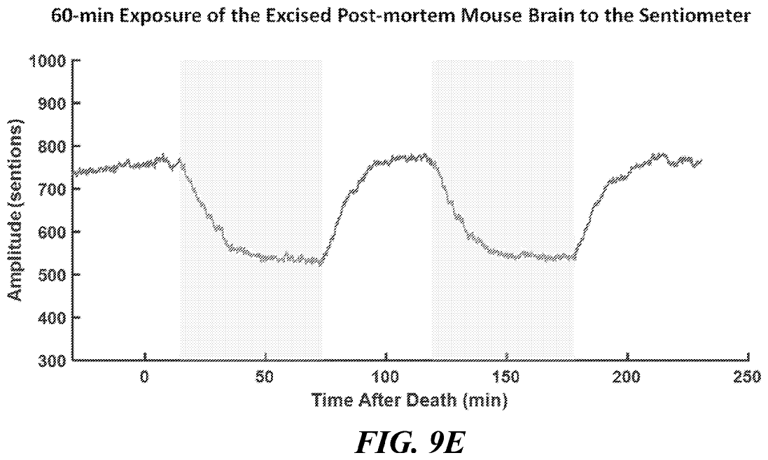

The study measured for somatic effects using animal subjects using a divergence/diffraction device (e.g., FIG. 3F). FIG. 9A shows measurements acquired from a person (primate) and an animal (rodent). FIG. 9B shows measurements acquired from invertebrate animals.

In the observation, invertebrates appear to show inverted responses. The highlighted regions in the plots of FIGS. 9A and 9B depict the duration of exposure to the animal.

Persistence and Alteration of the Response after Death in Mice

The study measured for somatic effects using animal subjects using a divergence/diffraction device after the death of the animal. FIG. 9C shows measured responses to 20-min exposure. The response appears to be inverted 2 hours after induction of euthanasia.

FIG. 9D shows a measured sentiometric response from an animal (euthanized mice) with a decapitated head and body. In FIG. 9D, the baseline measurement shows an inversion of the response being observed with respect to exposure of the head (before death).

FIG. 9E shows measured sentiometric response from an excised brain of an animal. In FIG. 9E, the excised brain was observed to produce an inverted response. It is hypothesized that a brain-dead patient would likely show an inverted response.

### Response to Other Body Parts

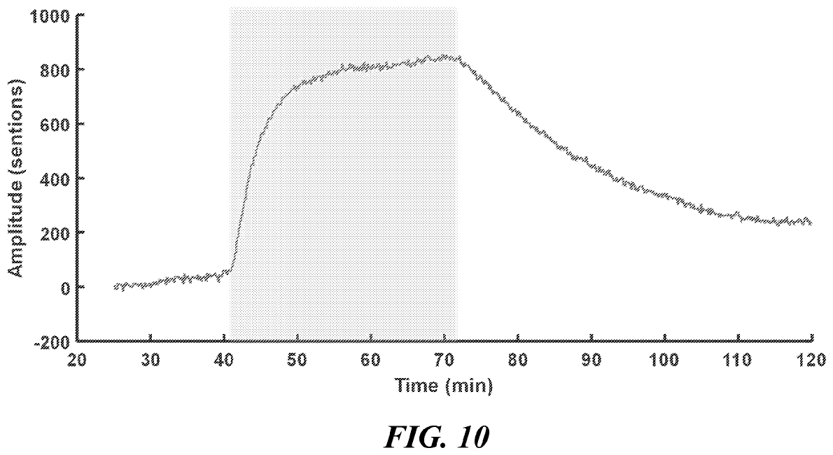

The study measured for somatic effects on body parts other than the brain or head. FIG. 10 shows measured sentiometric response induced by a 30-min exposure of the hand to a sensor module of a sentiometer placed 15 cm from the side of the body. FIG. 10 shows the effect likely being spread to the rest of the body. The pre-response baseline includes the effect at 15 cm. The highlighted region in the plot depicts the duration of exposure.

### Discussion

Developing a test capable of detecting consciousness is an important and ongoing area of research, and further insights into the basis of consciousness are of general and scientific interest. A well-established test would have wider applications for use in anesthesiology, diagnosis of altered states of consciousness, sleep medicine, diagnosis of psychiatric conditions, law enforcement and intelligence work, detection of consciousness in non-human organisms, and mapping of the neural code of conscious experience.

Currently, no reliable method has been developed to accurately assess, measure, and monitor the levels of consciousness in patients. Approaches involve the use of fMRI, EEG, TMS, and, in recent times, the combinatory use of the above-listed techniques and machine learning. Currently, there are two principal commercial EEG anesthesia monitors widely used: (1) The Bispectral index (BIS) from Aspect Medical Systems, Inc., USA and Covidien plc, Ireland, and (2) The “Entropy Module” from GE Healthcare, US. Both use EEG signaling and algorithmic processes to monitor various states of consciousness. However, these two have been observed to have varying degrees of success.

There is art in the literature (**Physics Essays, 2019) that uses the concept of the collapse of the wave function in quantum mechanics as a measure of mind over matter effects of conscious will. The current disclosure is built on the concept that an apparatus that can detect the conversion of light waves into photons should be able to confirm its two main predictions, namely: 1) that a conscious experience-producing neural activity focus located within a short distance of this apparatus should convert more light waves into photons; and 2) the temporal pattern of this conversion when detected as a signal above the baseline noise should represent the code for the corresponding experience.

Recently, Cleveland Medical Devices Inc. and Everest Biomedical Instruments, introduced its WAVCNS index (Wavelet-based Anesthetic Value for Central Nervous System), based on the company's NeuroSense monitor. Like BIS, WAV is based on the analysis of EEG signals recorded from surgical patients on a 0 to 100 scale. In a recent study, CleveMed investigators reported that WAVCNS outperformed BIS. The researchers attributed the performance difference to a 15-second time delay during induction that exists with BIS but not WAVCNS. BIS also uses unilateral monitoring, which CleveMed says hampers intra-patient reproducibility. Everest Biomedical Instruments also markets a system called SNAP II to help assess the level of consciousness. The SNAP index uses both high and low-frequency EEG evaluated in real-time.

### Example Computing System

It should be appreciated that the logical operations described above can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory 230, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document are hereby incorporated by reference in their entirety herein.
[1] van den Corput, Daniěl. “Locked in Syndrome Machine Learning Classification using Sentence Comprehension EEG Data.” arXiv e-prints (2020): arXiv-2006.
[2] Casali, Adenauer G., et al. “A theoretically based index of consciousness independent of sensory processing and behavior.” Science translational medicine 5.198 (2013): 198ra105-198ra105.
[3] Radin, Dean, et al. “Consciousness and the double-slit interference pattern: Six experiments.” Physics Essays 25.2 (2012): 157.
[4] KR102095898B1
[5] U.S. Ser. No. 10/799,134B2
[6] US20110118619A1
[7] The Bispectral index (BIS) from Aspect Medical Systems, Inc., USA and Covidien plc, Ireland.
[8] Depth of Anesthesia Monitoring Devices Market Size, Share, Growth, Report 2021-2030
[9] Global Depth of Anesthesia Monitoring Market—Industry Trends and Forecast to 2029
[10] Cavuoto, J., 2022., “Competition Heats Up in Consciousness Monitoring,” Neurotech Business Report.

What is claimed is:

1. A method for consciousness, unconsciousness, or sentience assessment, the method comprising:
providing a modular apparatus comprising a housing having located therein a photon source and a photonic sensor or sensor assembly, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas;
placing the modular apparatus in proximity to a person or animal;
measuring, via electric circuitries, electrical current corresponding to the received photons for each of the plurality of channels or areas while the apparatus is in proximity to a person or animal; and
outputting, via the electric circuitries or a computing device on a display, the measured electrical current or a parameter derived therefrom for each of the plurality of channels or areas, wherein the measured electrical current is employed as a measure or indicator of consciousness or sentience of the person or animal.

2. A method for consciousness, unconsciousness, or sentience assessment, the method comprising:
providing a modular apparatus comprising a photon source and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas;
placing the modular apparatus in proximity to a person or animal;
measuring, via electric circuitries, electrical current corresponding to the received photons for each of the plurality of channels or areas while the apparatus is in proximity to a person or animal;
determining, by a processor, a consciousness or sentience measure based on the measured electrical current or a parameter derived therefrom for at least one of the plurality of channels or areas exceeds a pre-defined threshold; and
outputting, via the electric circuitries or a computing device on a display, the consciousness or sentience measure, wherein the consciousness or sentience measure is employed as a measure or indicator of consciousness or sentience of the person or animal.

3. A method for consciousness, unconsciousness, or sentience assessment, the method comprising:
providing a plurality of modular apparatuses each comprising a photon source and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas, wherein the plurality of modular apparatuses are disposed at a plurality of locations on a headwear;
placing the plurality of modular apparatuses in proximity to a person or animal;
measuring, via electric circuitries, electrical current corresponding to the received photons for each of the plurality of channels or areas while the apparatus is in proximity to a person or animal;
determining, by a processor, a consciousness or sentience measure based on the measured electrical current or a parameter derived therefrom for at least one of the plurality of channels or areas exceeds a pre-defined threshold; and
outputting, via the electric circuitries or a computing device on a display, the consciousness or sentience measure, wherein the consciousness or sentience measure is employed as a measure or indicator of consciousness or sentience of the person or animal.

4. A method for consciousness, unconsciousness, or sentience assessment, the method comprising:
providing a first assembly of one or more modular apparatuses, including a first modular apparatus comprising a photon source and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas, wherein first modular apparatus is disposed at a location on a wearable device to be placed on person or animal;
providing a second apparatus comprising a photon source and a photonic sensor or sensor assembly housed in a body housing, wherein the photonic sensor or sensor assembly comprises at least one elongated area defining a plurality of channels or areas each configured to (i) receive interference pattern or divergent/diffracted waves of light emitted by the photon source and (ii) measure electrical current corresponding to the received photons for each of the plurality of channels or areas;
placing the first assembly comprising at least the first modular apparatus at a first position proximal to the person or animal;
placing the second apparatus at a second position proximal to the person or animal, wherein the first position is closer to the person or animal than the second position;
measuring, via electric circuitries, as a first measurement, electrical current corresponding to the received photons for each of the plurality of channels or areas of the first modular apparatus while the first assembly is located at the first position;
measuring, via electric circuitries, as a second measurement, electrical current corresponding to the received photons for each of the plurality of channels or areas of the second apparatus while the first assembly is located at the second position;
determining, by a processor, a consciousness or sentience measure based the first measurement and the second measurement; and
outputting, via the electric circuitries or a computing device on a display, the consciousness or sentience measure or an indicator derived therefrom, wherein the consciousness or sentience measure or the indicator is employed as a measure or indicator of consciousness or sentience of the person or animal.

5. The method of claim 1 or 2, wherein the modular apparatus is disposed on a head wear.

6. The method of claim 3 or 4, where the plurality of modular apparatuses includes at least one of: 2 apparatuses, 3 apparatuses, 4 apparatuses, 5 apparatuses, 6 apparatuses, 7 apparatuses, 8 apparatuses, 9 apparatuses, 10 apparatuses, between 10 and 16 apparatuses, between 16 and 32 apparatuses, and between 32 and 64 apparatuses.

7. The method of any one of claims 3-6, further comprising;
generating, by the processor or a different computing device, a visual output of a highest amplitude channel or a statistical parameter derived of the measurement, for each of the plurality of modular apparatuses.

8. The method of any one of claims 1-7, wherein the photonic sensor or sensor assembly comprises an array of photodiodes.

9. The method of any one of claims 1-8, wherein the photon source comprises one or more an LED, a laser, or an assembly thereof.

10. The method of any one of claims 1-9, wherein the measuring, via electric circuitries, electrical current corresponding to the received photons for each of the plurality of channels or areas comprises:
determining, by the processor or hardware circuitries, band regions for the received photons for each of the plurality of channels or areas.

11. The method of claim 4, wherein the first modular apparatus is identical in configuration to the second apparatus.

12. The method of claim 4, wherein the first modular apparatus has a first configuration, wherein second apparatus has a second configuration, and wherein the first configuration is different from the second configuration.

13. The method of any one of claims 1-12, wherein the output is used by a clinician or a machine to adjust or administer anesthesia.

14. The method of any one of claims 1-12, wherein the output is used by a clinician or a machine to identify or provide labels or classification among at least brain death, comatose, vegetative, minimally conscious, and locked-in states.

15. The method of any one of claims 1-12, wherein the output is used by a clinician or a machine to (i) quantify perception of pain or distress, (ii) quantify level or state of sleep (e.g., in a sleep study or sleep treatment), or (iii) quantify hallucination states, moods, beliefs, recurrent thoughts or recurrent feelings.

16. The method of any one of claims 1-15, wherein the modular apparatus includes a single or double slit located between the photon source and the photonic sensor or sensor assembly to generate the interference pattern.

17. The method of any one of claims 1-15, wherein the photonic sensor or sensor assembly of the modular apparatus is configured to measure the divergent/diffracted waves of light emitted by the photon source.

18. The method of any one of claims 1-17, wherein the at least one elongated area includes a horizontal elongated region.

19. The method of any one of claims 1-17, wherein the at least one elongated area includes a vertical elongated region.

20. A device or system comprising:
a modular apparatus comprising:
a housing having located therein a photon source,
a photonic sensor or sensor assembly, wherein the photonic sensor or sensor assembly comprises at least one elongated area defining a plurality of channels or areas each configured to receive interference pattern or divergent/diffracted waves of light emitted by the photon source; and
a controller configured with electrical circuitry to measure electrical current corresponding to the received photons for each of the plurality of channels or areas.

21. The device or system of claim 20, wherein the modular apparatus can be placed in proximity to a person or animal to provide a measure or indicator of consciousness or sentience of the person or animal.

22. The device or system of claim 20 or 21, further comprising features recited in any one of the methods of claims 1-19.

23. A device or system comprising:
a modular apparatus comprising:
a miniaturized Mach Zehnder interferometer; and
a controller configured with electrical circuitries to measure electrical current corresponding to the received photons of the miniaturized Mach Zehnder interferometer, wherein the controller is configured to output, via the electric circuitries or a computing device on a display, the measured electrical current or a parameter derived therefrom, wherein the measured electrical current is employed as a measure or indicator of consciousness or sentience of the person or animal.

24. A non-transitory computer-readable medium having instructions stored thereon, wherein execution of the instructions by a processor, causes the processor to (i) perform in whole or in part any one of the methods of claims 1-19 or (ii) operate in whole or in part any one of the device or system of claims 20-23.
